(19) United States
(12) Patent Application Publication
(10) Pub. No.: US 2023/0304954 A1
(43) Pub. Date: Sep. 28, 2023

(54) NOBLE METAL NANOPARTICLE-DECORATED ZINC OXIDE-ON-METAL GAS SENSOR FOR KETONE DETECTION

(71) Applicant: KING FAHD UNIVERSITY OF PETROLEUM AND MINERALS, Dhahran (SA)

(72) Inventors: Qasem Ahmed DRMOSH, Dhahran (SA); Yousif Ahmed AL-WAJIH, Dhahran (SA)

(73) Assignee: KING FAHD UNIVERSITY OF PETROLEUM AND MINERALS, Dhahran (SA)

(21) Appl. No.: 17/702,327

(22) Filed: Mar. 23, 2022

(51) Int. Cl. G01N 27/12; G01N 33/00

(52) U.S. Cl. CPC G01N 27/127; G01N 33/0047

(57) ABSTRACT

A gas sensor is provided. The gas sensor comprises a substrate and an active material, comprising first noble metal nanoparticles, a zinc oxide layer, and second noble metal nanoparticles. These components are arranged such that the zinc oxide layer prevents contact between the second noble metal nanoparticles and the first noble metal nanoparticles and between the second noble metal nanoparticles and the substrate. A method of forming the gas sensor is also provided. The method involves sputtering a first film of a first noble metal onto the substrate, annealing to form the first noble metal nanoparticles, depositing the zinc oxide layer, sputtering a second film of a second noble metal, and annealing to form the gas sensor. The gas sensor is used in a method of detecting the presence of a ketone in a gas sample.

### STATEMENT OF PRIOR DISCLOSURE BY THE INVENTORS

Aspects of this technology are described in the article “Engineering the depletion layer of Au-modified ZnO/Ag core-shell films for high-performance acetone gas sensing” published in Sensors & Actuators B. Chemical, 2021, 129851, available on Mar. 26, 2021, which is incorporated herein by reference in its entirety.

### BACKGROUND OF THE INVENTION

### Field of the Invention

The present disclosure relates to a gas sensor, a method of forming the gas sensor, and a method of sensing the presence of a ketone in a gas sample using the gas sensor.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

There currently exists a great need to develop simple, fast, sensitive, inexpensive, and non-invasive sensors for the accurate detection of harmful gases. Such gases may represent hazards present in the environment or an enclosed space or be indicative of diseases or disorders, particularly respiratory or oral diseases. Diagnosis through analysis of human exhaled breath offers a simple, inexpensive, non-invasive, low cost, and rapid detection.

A typical exhaled human breath comprises carbon dioxide, nitrogen, water, oxygen, and hundreds of other trace gases including ammonia and volatile organic compounds (VOCs), such as ethane, acetone, and isoprene [A. T. Güntner, et. al., ACS Sens., 2019, 4, 268-280; S. Jeong, et. al., Adv. Mater., 2020, 2002075; and W. M. Ahmed, et. al., ACS Infect. Dis., 2017, 3, 695-710]. Various diseases, disorders, and infections, such as lung cancer, asthma, diabetes, and the recent COVID-19 virus, have been correlated with changes in the trace gas makeup of human exhaled breath. For example, the acetone concentration differs in the breath of normal and diabetic patients. The presence of acetone in exhaled breath has been employed as a biomarker for diabetes mellitus. The level of acetone in diabetic patients ranges between 1.25 and 2.5 ppm, while in healthy people it is found to be between 0.2 and 1.8 ppm. Moreover, prolonged exposure to acetone has a negative impact. For instance, inhaling moderate to high levels of acetone can cause nausea, increased pulse rate, headaches, fatigue, and skin damage. In rare cases, prolonged exposure to acetone can damage vital human organs, such as the kidney, liver, and reproduction [A. Rydosz, J. Diabetes Sci. Technol., 2015, 9, 881-884]. Therefore, it is imperative to develop a fast, robust and inexpensive sensing method for routine acetone detection.

Different sensing techniques, such as optical sensors, electrochemical sensors, and acoustic waves, have been developed for acetone sensing [J. Wang, et. al., Sens. Actuators, B Chem., 2020, 321, 128489; M. Manjula, et. al., Appl. Phys. A Mater. Sci. Process., 2020, 126, 718; F. Nadeem, et. al., Sensors, 2018, 18, 2050; W. Liu, et. al., Sens. Actuators, B Chem., 2019, 298, 126871; I. C. Weber, et. al., Adv. Sci., 2020, 7, 2001503; and K. Xu, et. al., Opt. Mater. Express, 2019, 9]. Compared to these types of sensors, however, chemiresistive gas sensors based on semiconductors, noble metals, organic compounds, polymers, or their hybrids are a promising alternative route. In general, semiconductor metal oxides nanomaterials are advantageous for gas sensing applications because of low fabrication cost, non-toxicity, small dimension, dense surface site for gas adsorptions, and suitable operating temperature. A wide variety of preparation techniques, such as sol-gel, electrospinning, template-assisted growth, chemical vapor etc., have been used to obtain such nanostructures. However, most of these synthesis methods involve surfactants or templates for successive fabrication of semiconductor metal oxide nanostructures. Removal of organics from the structure is challenging and affects the reproducibility of analysis. Further, some semiconductor metal oxides suffer from disadvantageous electrical properties, making gas sensing difficult or inefficient. Since the gas must interact with the surface of a sensor, the depletion layer in the semiconductor plays a crucial role in determining the density of free electrons at the surface, and hence the sensor's performance. Therefore, modulation of the depletion layer may represent an avenue to improve the gas sensing properties of semiconductor metal oxide-based sensing materials.

Accordingly, it is an objective of the present disclosure to provide a semiconductor gas sensor with tailored surface electronic properties and a method of its fabrication.

### SUMMARY OF THE INVENTION

The present disclosure relates to a gas sensor, comprising a substrate and an active material, comprising first noble metal nanoparticles disposed on the substrate, a zinc oxide layer disposed on the first noble metal nanoparticles and the substrate, and second noble metal nanoparticles disposed on the zinc oxide layer, wherein the zinc oxide layer prevents contact between the second noble metal nanoparticles and the first noble metal nanoparticles and between the second noble metal nanoparticles and the substrate.

In some embodiments, the first noble metal nanoparticles have a mean particle size of 50 to 300 nm.

In some embodiments, the first noble metal nanoparticles are silver nanoparticles.

In some embodiments, the zinc oxide layer has a thickness of 1 to 75 nm.

In some embodiments, the zinc oxide layer comprises wurtzite zinc oxide which is crystalline by PXRD.

In some embodiments, the second noble metal nanoparticles have a mean particle size of 25 to 250 nm.

In some embodiments, the second noble metal nanoparticles are gold nanoparticles.

In some embodiments, the active material has a band gap of 2.80 to 3.20 eV.

The present disclosure also relates to a method of forming the gas sensor, the method comprising sputtering a first film of a first noble metal onto the substrate to form a first film-comprising material, annealing the first film-comprising material to form the first noble metal nanoparticles, depositing the zinc oxide layer on the first noble metal nanoparticles by sputtering, sputtering a second film of a second noble metal onto the zinc oxide layer to form a second film-comprising material, and annealing the second film-comprising material to form the gas sensor.

In some embodiments, the first noble metal is silver.

In some embodiments, the second noble metal is gold.

In some embodiments, the first film has a thickness of 1 to 75 nm, and the second film has a thickness of 1 to 50 nm.

In some embodiments, the first film-comprising material is annealed at 450 to 650° C. under inert atmosphere.

In some embodiments, the second film-comprising material is annealed at 500 to 700° C. under inert atmosphere.

The present disclosure also relates to a method of detecting the presence of a ketone in a gas sample, the method comprising applying a voltage to the gas sensor, exposing the gas sample to the gas sensor, and detecting a change in the electrical properties of the gas sensor to determine whether a ketone is present or absent in the gas sample. In some embodiments, the ketone has a molecular weight of less than 300 g/mol.

In some embodiments, the ketone is acetone.

In some embodiments, the exposing is performed at 25 to 300° C.

In some embodiments, the method has a lower detection limit of 0.1 to 1 ppm of ketone.

In some embodiments, the method has a lower detection limit of 0.1 to 1 ppm of acetone.

### BRIEF DESCRIPTION OF THE DRAWINGS

FIG. 1 shows a schematic depiction of the fabrication steps for the gas sensor.

FIG. 2 shows PXRD patterns of Ag thin film, Ag NPs, ZnO thin film, ZnO-on-Ag film, Au NPs, and Au/ZnO/Ag film.

FIGS. 3A-3D shows FESEM images of Ag film (FIG. 3A), Ag NPs (FIG. 3B), ZnO-on-Ag film (FIG. 3C), and Ag (NPs)/ZnO film/Au (NPs) thin films (FIG. 3D).

FIG. 4 shows a histogram of the size distribution of Ag nanoparticles.

FIG. 5 shows UV characteristics of 61 nm Ag thin films before and after annealing at 550° C.

FIG. 6 shows a histogram of the size distribution of Au nanoparticles on Au/ZnO/Ag film

FIGS. 7A-7D are EDX mapping images of Au/ZnO/Ag film, where FIG. 7A is a SEM image of the analyzed area, FIG. 7B is a mapping of Zn, FIG. 7C is a mapping of Au, and FIG. 7D is a mapping of Ag.

FIGS. 8A-8E show XPS spectra of the gas sensor, where FIG. 8A is a survey spectrum of ZnO, Ag/ZnO, and Au/ZnO/Ag films, and FIGS. 8B-8E are XPS core level spectra of the Ag 3d region (FIG. 8B), the Zn 2p region (FIG. 8C), the Au 4f region (FIG. 8D), and the O1s region (FIG. 8E) of the Au/ZnO/Ag films.

FIGS. 9A-9D are plots of the XPS depth profile of Au/ZnO/Ag film, where FIG. 9A shows the Au4f region, FIG. 9B shows the Zn2p region, FIG. 9C shows the Ag3d region, and FIG. 9D shows the O1s region.

FIG. 10A is a plot of the UV-vis absorption spectra of Ag film, Ag NPs, and Au/ZnO/Ag film.

FIG. 10B is a plot of the optical band gap calculation of ZnO and Au/ZnO/Ag film.

FIG. 11A is a plot of the response of the ZnO/Ag core-shell films at different ZnO thickness' and various for 1 ppm of acetone at 150° C.

FIG. 11B is a plot of response vs temperature for different concentrations of Au over the surface of 11 nm Ag/ZnO (13 nm) at different operating temperatures.

FIGS. 11C-11D show dynamic response-recovery curves toward 500 ppb, 1 ppm, 5 ppm, and 10 ppm of acetone for ZnO/Ag (FIG. 11C) and Au/ZnO/Ag (FIG. 11D) at 150° C.

FIG. 11E shows the response vs. acetone concentration of ZnO/Ag core-shell, and Au/ZnO/Ag sensors at 150° C. operating temperature.

FIG. 11F shows the repeatability test of the Au/ZnO/Ag sensor toward 10 ppm of acetone.

FIG. 11G is a plot of the response and recovery time of Au/ZnO/Ag sensor toward 10 ppm of acetone at 150° C.

FIG. 11H is a plot of the selectivity of the Au/ZnO/Ag sensor towards 5 ppm of acetone at 150° C.

FIG. 11I shows the long term stability of ZnO/Ag and Au/ZnO/Ag sensors toward 5 ppm acetone at 150° C.

FIG. 12 is a depiction of a plausible mechanism for acetone gas sensing in the presence of ZnO, Au/ZnO), and Au decorated ZnO-on-Ag sensor.

### DETAILED DESCRIPTION OF THE INVENTION

In the following description, it is understood that other embodiments may be utilized and structural and operational changes may be made without departure from the scope of the present embodiments disclosed herein.

### Definitions

As used herein the words “a” and “an” and the like carry the meaning of “one or more.”

As used herein, the terms “optional” or “optionally” means that the subsequently described event(s) can or cannot occur or the subsequently described component(s) may or may not be present (e.g., 0 wt. %).

As used herein, the term “agglomerates” refers to a clustered particulate composition comprising primary particles, the primary particles being aggregated together in such a way so as to form clusters thereof, at least 50 volume percent of the clusters having a mean size that is at least 2 times the mean size of the primary particles, and preferably at least 90 volume percent of the clusters having a mean size that is at least 5 times the mean size of the primary particles.

As used herein, the term “noble metal” refers to a metallic element selected from the group consisting of gold, platinum, palladium, ruthenium, rhodium, osmium, silver, copper, mercury, rhenium, iridium, and alloys thereof. Examples of copper alloys include, but are not limited to gilding metal; Muntz metal; beryllium copper; nickel silver; cupronickel; Dunce metal; bronzes such as manganese bronze, tin bronze, leaded tin bronze, aluminum bronze, silicon bronze, phosphor bronze, commercial bronze, architectural bronze, mild bronze, bell metal, arsenical bronze, speculum metal, and cymbal alloy; and brasses such as Abyssinian gold, admiralty brass, Aich's alloy, aluminum brass, arsenical brass, cartridge brass, common brass, DZR brass, delta metal, free machining brass, high brass, leaded brass, low brass, manganese brass, naval brass, nickel brass, Nordic gold, drichalcum, Prince's metal, red brass (also known as gunmetal), tombac, silicon tombac, tonval brass, and yellow brass. Other exemplary alloys include gold alloys with copper and silver (colored gold, crown gold, electrum), gold alloys with rhodium (rhodite), gold alloys with copper (rose gold, tumbaga), gold alloys with nickel and palladium (white gold), gold alloys including the addition of platinum, manganese, aluminum, iron, indium and other appropriate elements or mixtures thereof, silver alloys with copper (shibuichi, sterling silver, Tibetan silver, Britannia silver), silver alloys with copper and gold (goloid), silver alloys with copper and germanium (argentium sterling silver), silver alloys with platinum (platinum sterling), silver alloys with copper (silver graphite), silver alloys including the addition of palladium, zinc, iridium, and tin and other appropriate elements or mixtures thereof, platinum alloys with gold, platinum alloys with cobalt, platinum alloys with rare earth elements, and platinum alloys with nickel.

### Gas Sensor

According to a first aspect, the present disclosure relates to a gas sensor, comprising a substrate and an active material. The active material comprises first noble metal nanoparticles disposed on the substrate, a zinc oxide layer disposed on the first noble metal nanoparticles and on the substrate, and second noble metal nanoparticles disposed on the zinc oxide layer, wherein the zinc oxide layer prevents contact between the second noble metal nanoparticles and the first noble metal nanoparticles and between the second noble metal nanoparticles and the substrate.

In general, the first noble metal nanoparticles can be any shape known to one of ordinary skill in the art. Examples of suitable shapes the first noble metal nanoparticles may take include spheres, spheroids, lentoids, ovoids, solid polyhedra such as tetrahedra, cubes, octahedra, icosahedra, dodecahedra, hollow polyhedral (also known as nanocages), stellated polyhedral (both regular and irregular, also known as nanostars), triangular prisms (also known as nanotriangles), hollow spherical shells (also known as nanoshells), tubes (also known as nanotubes), nanosheets, nanoplatelets, nanodisks, rods (also known as nanorods), and mixtures thereof. In the case of nanorods, the rod shape may be defined by a ratio of a rod length to a rod width, the ratio being known as the aspect ratio. For first noble metal nanoparticles of the current invention, nanorods should have an aspect ratio less than 1000, preferably less than 750, preferably less than 500, preferably less than 250, preferably less than 100, preferably less than 75, preferably less than 50, preferably less than 25. Nanorods having an aspect ratio greater than 1000 are typically referred to as nanowires and are not a shape that the first noble metal nanoparticles are envisioned as having in any embodiments.

In some embodiments, the first noble metal nanoparticles have uniform shape. Alternatively, the shape may be non-uniform. As used herein, the term “uniform shape” refers to an average consistent shape that differs by no more than 10%, by no more than 5%, by no more than 4%, by no more than 3%, by no more than 2%, by no more than 1% of the distribution of first noble metal nanoparticles having a different shape. As used herein, the term “non-uniform shape” refers to an average consistent shape that differs by more than 10% of the distribution of first noble metal nanoparticles having a different shape. In one embodiment, the shape is uniform and at least 90% of the first noble metal nanoparticles are spherical or substantially circular, and less than 10% are polygonal. In another embodiment, the shape is non-uniform and less than 90% of the first noble metal nanoparticles are spherical or substantially circular, and greater than 10% are polygonal.

In some embodiments, the first noble metal nanoparticles have a mean particle size of 50 to 300 nm, preferably 55 to 287.5 nm, preferably 60 to 275 nm, preferably 65 to 262.5 nm, preferably 70 to 250 nm, preferably 75 to 237.5 nm, preferably 80 to 225 nm, preferably 85 to 212.5 nm, preferably 90 to 200 nm, preferably 95 to 187.5 nm, preferably 100 to 175 nm. In embodiments where the first noble metal nanoparticles are spherical, the particle size may refer to a particle diameter. In embodiments where the first noble metal nanoparticles are polyhedral, the particle size may refer to the diameter of a circumsphere. In some embodiments, the particle size refers to a mean distance from a particle surface to particle centroid or center of mass. In alternative embodiments, the particle size refers to a maximum distance from a particle surface to a particle centroid or center of mass. In some embodiments where the first noble metal nanoparticles have an anisotropic shape such as nanorods, the particle size may refer to a length of the nanorod, a width of the nanorod, an average of the length and width of the nanorod. In some embodiments in which the first noble metal nanoparticles have non-spherical shapes, the particle size refers to the diameter of a sphere having an equivalent volume as the particle. In some embodiments in which the first noble metal nanoparticles have non-spherical shapes, the particle size refers to the diameter of a sphere having an equivalent diffusion coefficient as the particle.

In some embodiments, the first noble metal nanoparticles of the present disclosure are monodisperse, having a coefficient of variation or relative standard deviation, expressed as a percentage and defined as the ratio of the particle size standard deviation (σ) to the particle size mean (μ) multiplied by 100 of less than 25%, preferably less than 10%, preferably less than 8%, preferably less than 6%, preferably less than 5%, preferably less than 4%, preferably less than 3%, preferably less than 2%. In some embodiments, the first noble metal nanoparticles of the present disclosure are monodisperse having a particle size distribution ranging from 80% of the average particle size to 120% of the average particle size, preferably 90-110%, preferably 95-105% of the average particle size. In some embodiments, the first noble metal nanoparticles are not monodisperse.

In general, the particle size may be determined by any suitable method known to one of ordinary skill in the art. In some embodiments, the particle size is determined by powder X-ray diffraction (PXRD). Using PXRD, the particle size may be determined using the Scherrer equation, which relates the full-width at half-maximum (FWHM) of diffraction peaks to the size of regions comprised of a single crystalline domain (known as crystallites) in the sample. In some embodiments, the crystallite size is the same as the particle size. For accurate particle size measurement by PXRD, the particles should be crystalline, comprise only a single crystal, and lack non-crystalline portions. Typically, the crystallite size underestimates particle size compared to other measures due to factors such as amorphous regions of particles, the inclusion of non-crystalline material on the surface of particles such as bulky surface ligands, and particles which may be composed of multiple crystalline domains. In some embodiments, the particle size is determined by dynamic light scattering (DLS). DLS is a technique which uses the time-dependent fluctuations in light scattered by particles in suspension or solution in a solvent, typically water to measure a size distribution of the particles. Due to the details of the DLS setup, the technique measures a hydrodynamic diameter of the particles, which is the diameter of a sphere with an equivalent diffusion coefficient as the particles. The hydrodynamic diameter may include factors not accounted for by other methods such as non-crystalline material on the surface of particles such as bulky surface ligands, amorphous regions of particles, and surface ligand-solvent interactions. Further, the hydrodynamic diameter may not accurately account for non-spherical particle shapes. DLS does have an advantage of being able to account for or more accurately model solution or suspension behavior of the particles compared to other techniques. In some embodiments, the particle size is determined by electron microscopy techniques such as scanning electron microscopy (SEM) or transmission electron microscopy (TEM).

In general, the shape description above may apply to the second noble metal nanoparticles. In some embodiments, the second noble metal nanoparticles have substantially the same shape as the first noble metal nanoparticles. In some embodiments, the second noble metal nanoparticles have a different shape from the first noble metal nanoparticles.

In some embodiments, the second noble metal nanoparticles have a mean particle size of 25 to 250 nm, preferably 30 to 225 nm, preferably 35 to 200 nm, preferably 40 to 175 nm, preferably 45 to 162.5 nm, preferably 50 to 150 nm, preferably 55 to 137.5 nm, preferably 60 to 125 nm, preferably 65 to 112.5 nm, preferably 70 to 100 nm. The particle size may be determined as described above.

In some embodiments, the second noble metal nanoparticles of the present disclosure are monodisperse, having a coefficient of variation or relative standard deviation, expressed as a percentage and defined as the ratio of the particle size standard deviation (a) to the particle size mean (0 multiplied by 100 of less than 25%, preferably less than 10%, preferably less than 8%, preferably less than 6%, preferably less than 5%, preferably less than 4%, preferably less than 3%, preferably less than 2%. In some embodiments, the second noble metal nanoparticles of the present disclosure are monodisperse having a particle size distribution ranging from 80% of the average particle size to 120% of the average particle size, preferably 90-110%, preferably 95-105% of the average particle size. In some embodiments, the second noble metal nanoparticles are not monodisperse.

In some embodiments, the first noble metal nanoparticles are formed from the same noble metal as the second noble metal nanoparticles. In preferred embodiments, the first noble metal nanoparticles are formed from a different noble metal as the second noble metal nanoparticles. For example, the first noble metal nanoparticles may be any noble metal as described above (gold, platinum, palladium, ruthenium, rhodium, osmium, silver, copper, mercury, rhenium, iridium, or an alloy thereof), and the second noble metal nanoparticles may be any other noble metal. Such a different noble metal may be a different alloy which comprises one or more noble metals in common, but which differs in terms of composition such as identity of other metals not in common or in the relative amounts of constituent metals. In some embodiments, the first noble metal nanoparticles are silver nanoparticles. In some embodiments, the second noble metal nanoparticles are gold nanoparticles. In preferred embodiments, the first noble metal nanoparticles are silver nanoparticles and the second noble metal nanoparticles are gold nanoparticles. In some embodiments, the first noble metal nanoparticles are crystalline by PXRD. In some embodiments, the second noble metal nanoparticles are crystalline by PXRD.

In some embodiments, the zinc oxide layer has a thickness of 1 to 75 nm, preferably 2 and 70 nm, preferably 3 and 65 nm, preferably 5 and 60 nm, preferably 7.5 and 55 nm, preferably 10 and 50 nm. In some embodiments, the zinc oxide layer comprises wurtzite zinc oxide which is crystalline by PXRD. In some embodiments, the zinc oxide is present in the zinc oxide layer as particles. Such particles may have a mean size of 1 to 75 nm, preferably 2 and 70 nm, preferably 3 and 65 nm, preferably 5 and 60 nm, preferably 7.5 and 55 nm, preferably 10 and 50 nm. Such particles may have a particle shape as described above. In some embodiments, the particles are present in the zinc oxide layer as agglomerates. In some embodiments, the agglomerates have a mean size of 1 to 75 nm, preferably 2 and 70 nm, preferably 3 and 65 nm, preferably 5 and 60 nm, preferably 7.5 and 55 nm, preferably 10 and 50 nm. In such embodiments, the agglomerates may have primary particles which have a mean particle size of 50%, preferably 45%, preferably 40%, preferably 35%, preferably 30%, preferably 25%, preferably 20%, preferably 15% of the agglomerate size.

In some embodiments, the active material has a band gap of 2.80 to 3.20 eV, preferably 2.85 to 3.175, preferably 2.90 to 3.15, preferably 2.95 to 3.125, preferably 3.0 to 3.1 eV.

### Method of Forming Gas Sensor

The present disclosure also relates to a method of forming the gas sensor, the method comprising sputtering a first film of a first noble metal onto the substrate to form a first film-comprising material, annealing the first film-comprising material to form the first noble metal nanoparticles, depositing the zinc oxide layer on the first noble metal nanoparticles by sputtering, sputtering a second film of a second noble metal onto the zinc oxide layer to form a second film-comprising material, and annealing the second film-comprising material to form the gas sensor.

In some embodiments, the first film is deposited by DC sputtering. In some embodiments, the DC sputtering of the first film is performed with power of 10 to 50 W, preferably 15 to 45 W, preferably 20 to 40 W, preferably 25 to 35 W, preferably 27.5 to 32.5 W, preferably 30 W. In some embodiments, the second film is deposited by DC sputtering. In some embodiments, the DC sputtering of the second film is performed with a power of 5 to 35 W, preferably 10 to 30 W, preferably 12.5 to 27.5 W, preferably 15 to 25 W, preferably 17.5 to 22.5 W, preferably 20 W. In some embodiments, the zinc oxide layer is deposited by DC reactive sputtering.

In some embodiments, the first noble metal is the same noble metal as the second noble metal. In preferred embodiments, the first noble metal is a different noble metal as the second noble metal. For example, the first noble metal may be any noble metal as described above (gold, platinum, palladium, ruthenium, rhodium, osmium, silver, copper, mercury, rhenium, iridium, or an alloy thereof), and the second noble metal may be any other noble metal. Such a different noble metal may be a different alloy which comprises one or more noble metals in common, but which differs in terms of composition such as identity of other metals not in common or in the relative amounts of constituent metals. In some embodiments, the first noble metal is silver. In some embodiments, the second noble metal is gold. In preferred embodiments, the first noble metal is silver and the second noble metal is gold.

In some embodiments, the first film has a thickness of 1 to 75 nm, preferably 2 to 65 nm, preferably 3 to 55 nm, preferably 4 to 45 nm. In some embodiments, the second film has a thickness of 1 to 50 nm, preferably 2 to 40 nm, preferably 3 to 34 nm.

In some embodiments, the first film-comprising material is annealed at 450 to 650° C., preferably 460 to 640° C., preferably 470 to 630° C., preferably 480 to 620° C., preferably 490 to 610° C., preferably 500 to 600° C., preferably 510 to 590° C., preferably 520 to 580° C., preferably 530 to 570° C., preferably 540 to 560° C., preferably 550° C. In preferred embodiments, the first film-comprising material is annealed under inert atmosphere. Such an inter atmosphere may be provided by any suitable inert gas, such as nitrogen, helium, argon, neon, and the like.

In some embodiments, the second film-comprising material is annealed at 500 to 700° C., preferably 510 to 690° C., preferably 520 to 680° C., preferably 530 to 670° C., preferably 540 to 660° C., preferably 550 to 650° C., preferably 560 to 640° C., preferably 570 to 630° C., preferably 580 to 620° C., preferably 590 to 610° C., preferably 600° C. In preferred embodiments, the annealing is performed under inert atmosphere.

### Method of Detecting the Presence of a Ketone

The present disclosure also relates to a method of detecting the presence of a ketone in a gas sample, the method comprising applying a voltage to the gas sensor, exposing the gas sample to the gas sensor, and detecting a change in the electrical properties of the gas sensor to determine whether a ketone is present or absent in the gas sample. In some embodiments, the change in the electrical properties of the gas sensor is a change in the resistance of the gas sensor. In some embodiments, the resistance is decreased in the presence of the ketone compared to in the absence of the ketone. In some embodiments, the resistance is increased in the presence of the ketone compared to in the absence of the ketone. In some embodiments, the presence of the ketone can be detected by a minimum change in the electrical properties. Such a minimum change may be referred to as a “threshold response”. That is, the change must be of a certain threshold magnitude to be considered a response which detects the presence of the ketone. In some embodiments, the threshold response is a change in the resistance of at least 40%, preferably at least 50%, preferably at least 60%, preferably at least 70%, preferably at least 80% of a resistance in the absence of the ketone.

In some embodiments, the method has a lower detection limit of 0.1 to 1 ppm, preferably 0.15 to 0.9 ppm, preferably 0.2 to 0.8 ppm, preferably 0.25 to 0.75 ppm, preferably 0.3 to 0.7 ppm, preferably 0.35 to 0.65 ppm, preferably 0.4 to 0.6 ppm, preferably 0.45 to 0.55 ppm, preferably 0.5 ppm of ketone. The lower detection limit refers to a minimum concentration of the ketone in the gas sample which causes a threshold response of the gas sensor. In some embodiments, the method has a maximum concentration of greater than 10,000 ppm, preferably greater than 7,500 ppm, preferably greater than 5,000 ppm, preferably greater than 2,500 ppm, preferably greater than 1,000 ppm, preferably greater than 750 ppm, preferably greater than 500 ppm, preferably greater than 250 ppm, preferably greater than 100 ppm of ketone.

In some embodiments, the ketone has a molecular weight of less than 300 g/mol. Examples of ketones having a molecular weight less than 300 g/mol include, but are not limited to acetone, 2-butanone, 2-pentanone, 3-pentanone, 2-hexanone, 3-hexanone, 2-heptanone, 3-heptanone, 2-octanone, 3-octanone, 4-octanone, cyclohexanone, acetophenone, 4-Phenylbutan-2-one, benzophenone, diacetyl, acetylacetone, cyclobutanone, muscone, chloroacetone, and camphor. The ketone may be a diketone. The diketone may be a 1,2-diketone, a 1,3-diketone, a 1,4-diketone, or any other suitable such diektone. In some embodiments, the ketone may have other, non-ketone functional groups present. Examples of such non-ketone functional groups include, but are not limited to other oxygen-containing functional groups, such as alcohol groups, carboxyl groups, and ether groups; nitrogen-containing functional groups such as amine groups, imine groups, and amide groups; alkene functional groups, alkyne functional groups; organohalide groups, and thiol groups. In some embodiments, the ketone is devoid of other oxygen-containing functional groups, such as alcohol groups, carboxyl groups, or ether groups. In some embodiments, the ketone is devoid of nitrogen-containing functional groups such as amine groups, imine groups, or amide groups. In some embodiments, the ketone is devoid of alkene or alkyne functional groups. In some embodiments, the ketone is devoid of organohalide functional groups. In some embodiments, the ketone is devoid of thiol functional groups.

In some embodiments, the ketone is acetone. In some embodiments, the method has a lower detection limit of 0.1 to 1 ppm, preferably 0.15 to 0.9 ppm, preferably 0.2 to 0.8 ppm, preferably 0.25 to 0.75 ppm, preferably 0.3 to 0.7 ppm, preferably 0.35 to 0.65 ppm, preferably 0.4 to 0.6 ppm, preferably 0.45 to 0.55 ppm, preferably 0.5 ppm of acetone. In some embodiments, the method has a maximum concentration of greater than 10,000 ppm, preferably greater than 7,500 ppm, preferably greater than 5,000 ppm, preferably greater than 2,500 ppm, preferably greater than 1,000 ppm, preferably greater than 750 ppm, preferably greater than 500 ppm, preferably greater than 250 ppm, preferably greater than 100 ppm of acetone.

In general, the exposing of the gas sample to the gas sensor may be performed in any suitable manner and with any suitable hardware known to one of ordinary skill in the art. For example, the gas sample may be flowed over the gas sensor. Such a flow may be at any suitable flow rate to achieve contact between the gas sample and the gas sensor. In some embodiments, the gas sample may be introduced into a chamber housing the gas sensor. Once introduced into the chamber, the gas sample may remain in the chamber without addition or removal of the gas sample for a measurement time. Throughout the measurement time, the gas sample may remain substantially static or may be agitated or stirred. In some embodiments, the gas sample is pre-treated prior to contact with the gas sensor. For example, the gas sample may be pre-heated, or may have one or more components removed, such as water vapor by passing the gas sample over a suitable drying agent.

In some embodiments, the gas sample comprises exhaled lung contents of an animal or human.

In some embodiments, the exposing is performed at 25 to 300° C., preferably 50 to 250° C., preferably 75 to 225° C., preferably 100 to 200° C., preferably 110 to 190° C., preferably 120 to 180° C., preferably 130 to 170° C., preferably 140 to 160° C., preferably 150° C.

In some embodiments, the method may include calculating a concentration of the ketone from the gas sample by using a magnitude of the changes in the electric current. Such changes may be caused by changes in the resistance of the gas sensor. The change in the resistance of the gas sensor may be due to interaction of the ketone with the gas sensor, particularly the second metal nanoparticles and/or the zinc oxide layer. This interaction may be any suitable type of interaction known to one of ordinary skill in the art. This interaction may be a specific chemisorption interaction which forms chemical bonds or involves a chemical reaction between the ketone and the gas sensor surface. Such bonds may be covalent or noncovalent. Alternatively, this interaction may be a physisorption interaction, for example electrostatic interactions such as ion (or charged species in general)-ion interactions, ion-dipole interactions, or dipole-dipole interactions; and Van der Waals interactions. Preferably, this interaction is reversible, allowing a desorption or other release of the ketone from the gas sensor. Preferably, this interaction does not permanently alter the chemical makeup or physical properties of the gas sensor after such desorption or release of the ketone from the gas sensor.

In some embodiments, the method shows a response (threshold change in the electrical properties of the gas sensor) only in the presence of a ketone. That is, the presence of other gases in the gas sample which are not a ketone do not cause a response in the gas sensor. Examples of such other gases which may be present which do not causes a response in the gas sample include, but are not limited to nitrogen, argon, helium, carbon dioxide, nitrogen dioxide, oxygen, hydrogen, ammonia, saturated hydrocarbons such as methane, ethane, propane, and butane, unsaturated hydrocarbons such as ethylene and acetylene, and water vapor. In this way, the presence of the ketone may be specifically detected by a response in the gas sensor.

The examples below are intended to further illustrate protocols for preparing and characterizing the gas sensor, as well as performing the method of detecting the presence of a ketone, and are not intended to limit the scope of the claims.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

### EXAMPLES

### Fabrication of Au/ZnO/Ag Thin Films

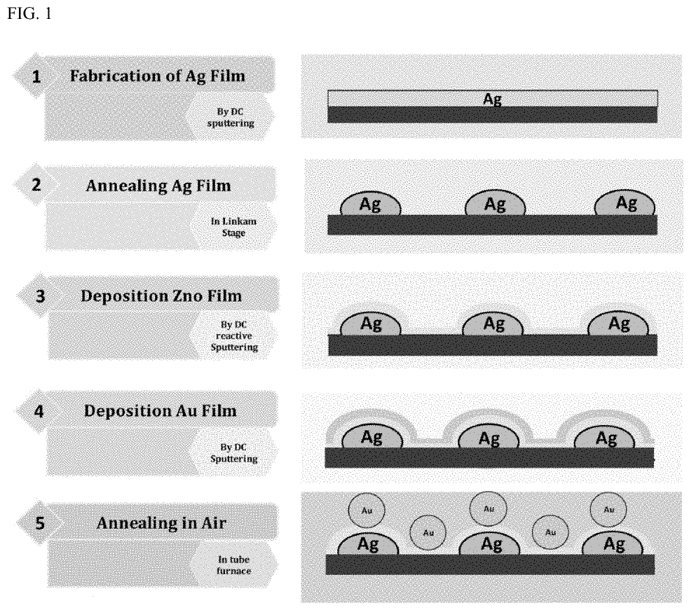

FIG. 1 shows the steps involved in the preparation of Au NPs decorated ZnO-on-Ag NP films. The overall fabrication procedure can be broadly divided into three different steps.

### —Fabrication of Well-Dispersed Ag Nanoparticles

Synthesis of Ag nanoparticles (NPs) on the surface of the interdigitated electrodes (IDE) was carried out by DC sputtering. Prior to sputtering, the Ag target (ACI alloy, Inc) was cleaned by pre-sputtering for 2 min. The base pressure, working pressure, and deposition power were adjusted to 2.4×10⁻⁵ torr, 2.8×10⁻³ torr, and 30 W, respectively. Four sets of Ag films with varying film thickness, such as 4 nm, 11 nm, 24 nm, and 45 nm, were prepared on the IDE. The as-prepared Ag films were then heated at 550° C. in Linkam stage for 1 h under a nitrogen environment. The process produced a film with well-dispersed Ag NPs.

### —Preparation of ZnO Film

The as-prepared Ag films were used to obtain ZnO-on-Ag NP films. Preparation of ZnO-on-Ag NP films was carried out as follows. Zinc metal was converted into ZnO thin film by DC reactive sputtering and deposited on the surface of Ag NPs film. Prior to ZnO disposition, the base pressure of the sputtering chamber was adjusted to about 1×10⁻⁵ torr, while the working pressure and magnetron power were 4.6×10⁻³ torr, and 100 W, respectively. The thickness of the ZnO layers which include 3, 13, 29, 34, 46, and 61 nm, was fine-tuned by adjusting the deposition time between 2 and 40 min.

### —Preparation of Au Decorated ZnO-On-Ag NP Films

The as-obtained ZnO-on-Ag NP films were further modified with Au by DC sputtering. Like other targets, the Au target (ACI alloy, Inc) was cleaned by pre-sputtering for 2 min. The base and working pressures were kept at about 1.5×10⁻⁵ torr and 3.3×10⁻³ torr, respectively. In order to investigate the effect of Au NPs on the acetone gas sensing properties, ultrathin films of Au were deposited on the IDE using 20 W for 5, 15, 25, 35, 45 s. Like ZnO films, the Au films with varying thicknesses of 4, 11, 24, and 45 nm were obtained by changing the deposition time. The as-prepared Ag/ZnO/Au electrodes were then heated at 600° C. for 1 h in a tube furnace under nitrogen to convert the Au layer into well-dispersed Au NPs.

### Gas Sensing Measurements

The as-fabricated electrodes, such as ZnO, Ag/ZnO, and Ag/ZnO/Au, were aged at 150° C. for 2 h to improve the stability of gas sensing material. The gas sensing properties were evaluated for various concentrations of acetone at different operating temperatures (RT to 300° C.). The change in electrical resistance, in the presence of acetone (Rg) and air (Ro), was followed by a semiconductor device analyzer (SDA; model Keysight B1500A, USA). The schematic of the gas sensing set-up used in this study is shown in FIG. 1. At first, the fabricated sensor was fixed into the gas sensing chamber (Linkam stage model: HFS600E-PB4). The two ends of the IDE were electrically connected to two counter pins of the SDA. Dry air entered through a mass flow controller (MFC) connected to the Linkam stage and used as the purging gas. The flow rates of the acetone and the air were kept constant at 100 standard cm3 per minute (sccm). The response (S) of the acetone sensors is defined according to Equation 1:

$$S ( \% ) = \frac{{R}_{g} - {R}_{a}}{{R}_{a}} \times 100 = \frac{Δ R}{{R}_{a}} \times 100 \qquad ( 1 )$$

### Characterization Methods of the Fabricated Devices

The crystal structures of the fabricated films were studied by powder X-ray diffraction (XRD, Rigaku MiniFlex) with Cu Kα radiation (λ=0.154178 nm) at 40 kV and 40 mA. A field-emission scanning electron microscope (FESEM, Tescan Lyra-3) was employed to examine the shape and size of the fabricated thin films. The surface compositions and the changes in binding states were investigated by X-ray Photoelectron Spectroscopy (XPS, ESCALAB250Xi, Thermo Fisher Scientific). Optical transmittance was determined by UV/Vis spectrophotometer (Jasco V-570, Japan) in the wavelength range of 300-1200 nm.

### Characterization Results

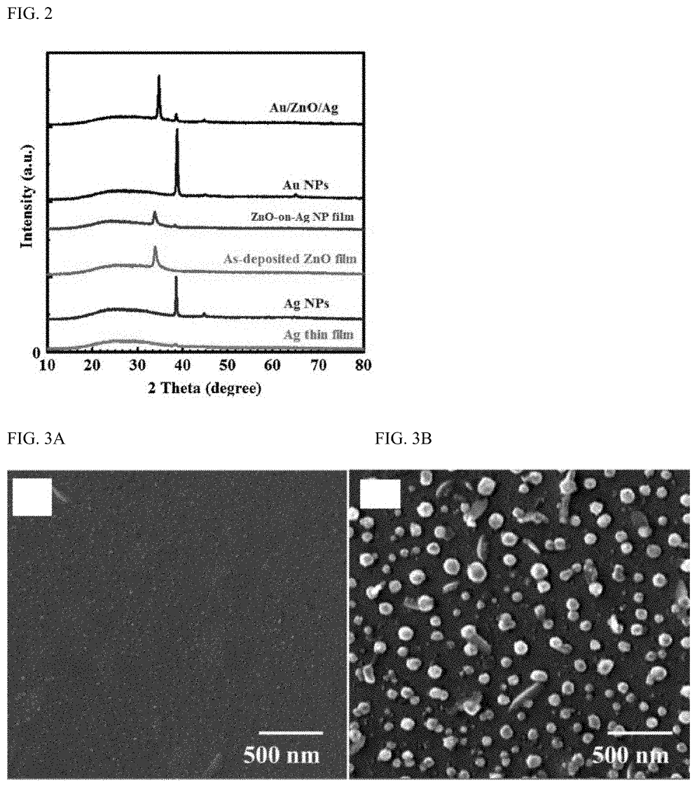

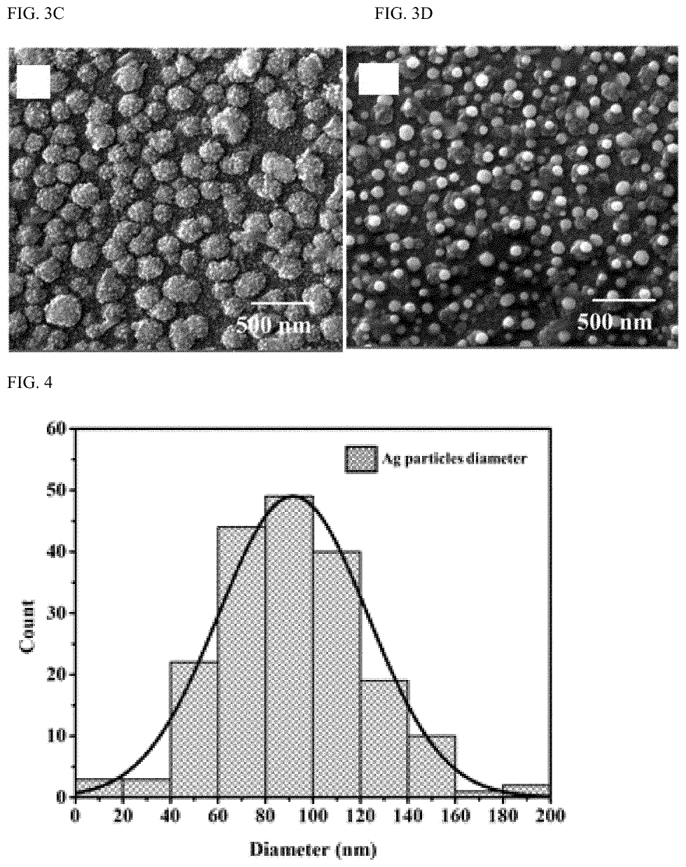

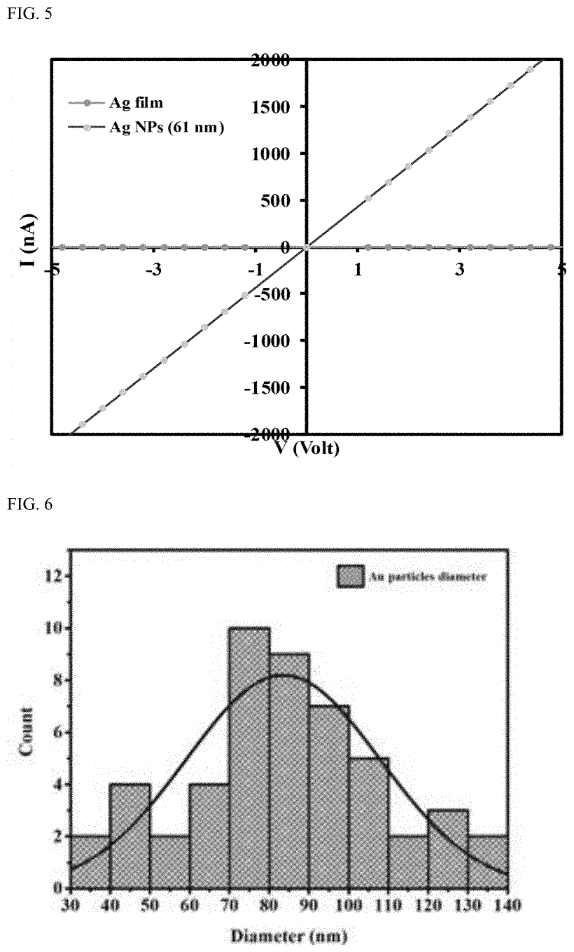

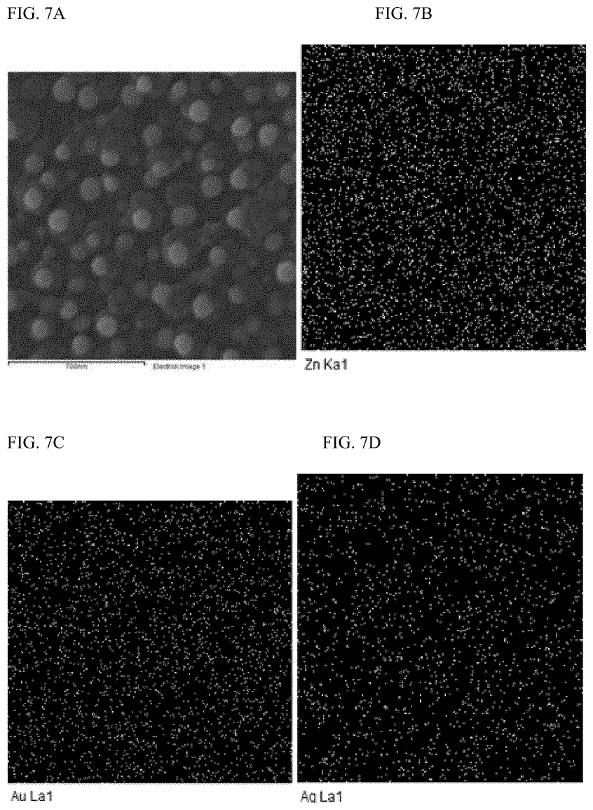

FIG. 2 shows the XRD patterns of Ag thin film, Ag NPs, pristine ZnO thin film, ZnO-on-Ag NP film, Au NPs, and Au/ZnO/Ag (Au—12 nm, ZnO—13 nm, Ag—11 nm). The Ag thin film shows a weak reflection at 38.3°, which is ascribed to (111), while Ag NPs possess two diffraction peaks at 38.54° and 44.60° corresponding to (111) and (200), respectively. The diffraction patterns of the ZnO film confirms its hexagonal wurtzite structure with preferred orientation along (002) direction, which is attributed to the lowest surface energy of the (002) compared with other directions [Z. L. Tseng, et. al., Sci. Rep., 2015, 5, 1-10, incorporated herein by reference in its entirety]. Three diffractions at 38.8°, 44.9°, and 65.1° corresponding to (111), (200), and (220) planes are perceptible in the case of the Au NPs (JCPDS standard of No. 04-0784). In the case of Au/ZnO/Ag, the XRD shows three patterns centered at 34.6°, 38.5°, and 44.6° corresponding to (002) of ZnO, (111) of Au and/or Ag, and (200) of Au and/or Ag, respectively. The (002) plane of ZnO has been shifted towards a higher angle after annealing at 600° C., which is presumably attributed to the stress relaxation due to the heating [M. A. Gondal, et. al., Appl. Surf. Sci., 2009, 256, 298-304, incorporated herein by reference in its entirety]. FIGS. 3A-3D show FESEM images of the fabricated samples Ag film, Ag NPs, ZnO-on-Ag NP film, and Au/ZnO/Ag thin film. The surface of the Ag film (FIG. 3A) showed dense, smooth, continuous, and homogenous film without any voids or cracks. When the Ag film was annealed at 550° C., the film morphology was transformed into nanoparticles with a size in the range of 70-250 nm (FIG. 3B). The process also led to the enhancement of the film crystallinity, which is consistent with the improvement in crystallinity indicated by the XRD patterns. The Ag particle size distribution, determined from 41 discrete Ag NPs with Image J software, is presented in FIG. 4. FIG. 5 compares the I-V measurements of the Ag thin films and the Ag NPs. The electrical resistivity of the Ag NPs was found to be much higher than that of Ag film, indicating a morphological and structural evolution of the Ag layer into Ag NPs. The FESEM image of the ZnO-on-Ag NP film (FIG. 3C) indicates that the surface morphology and shape were similar to that of the Ag particle. The microscopic image of annealed Au/ZnO/Ag film, FIG. 3D, shows the Au NPs were found to be mainly formed at the top of the ZnO-on-Ag NP film with few on the ZnO layer only. The mean size of Au NPs (finer bright) is estimated to be between 50 and 150 nm, as calculated from more than 200 individual NPs. The particle size histogram is presented in FIG. 6. Elemental mapping (EDX) images are shown in FIGS. 7A-7D showing the distribution of Zn, Au, and Ag in the Au/ZnO/Ag film.

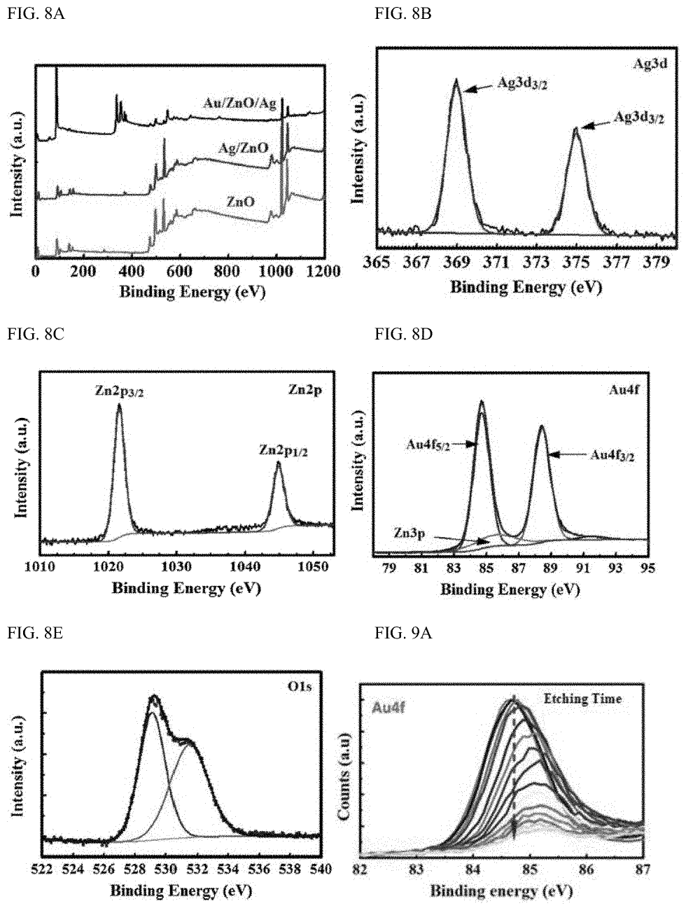

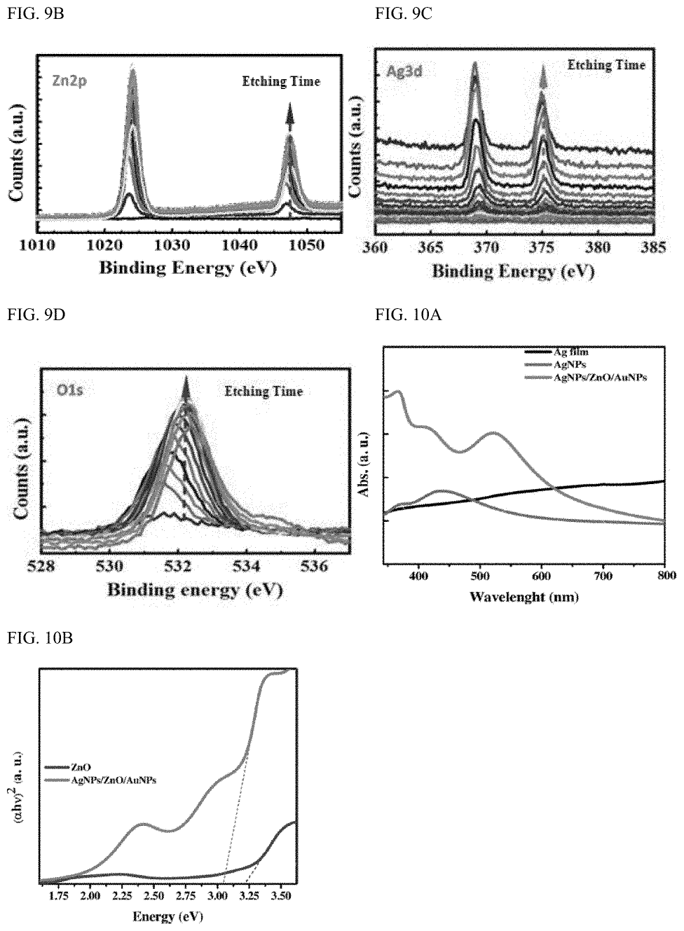

Surface chemical composition and oxidation state were examined by X-ray photoelectron spectroscopy (XPS). The survey spectra of ZnO thin film, ZnO-on-Ag NP film, and Au/ZnO/Ag along with fine scans of the elemental lines of the Au/ZnO/Ag sample are shown in FIG. 8A. The binding energies of all spectra are calibrated by C1s, which originates from the absorbed CO₂. The survey XPS spectra of the ZnO film and the ZnO/Ag consist of Zn, and O, and Zn, O, and Ag, respectively; while the survey XPS spectrum of the Au/ZnO/Ag possesses Zn, O, Ag, and Au. No trace of other elements was observed in the three spectra, confirming the high purity of the fabricated films. In FIG. 8B, the two major emission peaks of Ag at around 369.08 and 375.07 are assigned to Ag 3d_{3/2} and Ag 3d_{5/2}, respectively, corresponding to Ag⁰ electronic state. These two peaks are shifted significantly compared with that of pure Ag (Ag 3d_{5/2}: 368.2 eV; Ag 3d_{3/2}: 374.2 eV), which is attributed to the modification in the surface state of ZnO in the ZnO/Ag system [Y. Cai, et. al., Colloids Surf. A Physicochem. Eng. Asp., 2013, 436, 787-795, incorporated herein by reference in its entirety]. The Fermi level (E_{f}) of ZnO is lower than that of Ag. When the ZnO is deposited onto Ag NPs, the new E_{f} of the composite material is formed which is between the E_{f} of ZnO and the E_{f} of Ag. Thus, electrons may drift from Ag to ZnO, which in turn leads to a shift in the Ag 3d binding energy to a lower value [J. Liu, et. al., ACS Sustain. Chem. Eng., 2019, 7, 11258-11266, incorporated herein by reference]. For the Au4f region, two photoelectric characteristic peaks at 84.76 and 88.50 eV are attributed to the 4f_{5/2} and 4f_{3/2} of the Au with spin-orbit splitting energy of 3.74 eV. These data were fitted using Gaussian/Lorenzain mixed with Shirley peak background using 50 iterations and 0.01 convergence. The background average at start and end was 0.5 eV. The high-resolution Zn 2p spectrum (FIG. 8D) showed two characteristic peaks located at 1022.01 eV (2p_{3/2}) and 1045.11 eV (2p_{1/2}) with spin-orbit splitting energy of 23.10 eV, suggesting the presence of Zn²⁺ form. A remarkable shift in the binding energies of Zn2p_{3/2} and Zn2p_{1/2}, as compared to pristine ZnO, to lower values, indicated a strong interaction between Au and ZnO. The E_{f} of ZnO is higher than that of Au, and hence electrons transfer from ZnO to Au is likely. Such a process can have a significant impact on the binding energy of the ZnO [P. Fageria, et. al., RSC Adv., 2014, 4, 24962-24972, incorporated herein by reference in its entirety]. The O1s peak in ZnO is often deconvoluted into three different peaks representing three different environments: (1) O₂⁻ ions in the wurtzite ZnO structure at low binding energies (529.50-530.7 eV), (2) oxygen vacancies at medium binding energies (530.7-531.7 eV), and (3) OH⁻ or any other surface adsorbed oxygen species at high binding energies (532.5-532.8 eV) [Q. A. Drmosh, et. al., Sens. Actuators, B Chem., 2017, 248, 868-877, incorporated herein by reference in its entirety]. The O1s XPS peak at about 529.60 eV in FIG. 8E is assigned to oxygen atoms in the ZnO lattice while the peak at 531.49 eV is attributed to the presence of oxygen vacancy defects. The shift in the binding energy of the Au/ZnO/Ag film, because of the diffusion of the carrier charges, is confirmed by the XPS depth profiling. To achieve this, the surface of the sample was sequentially etched using a 2 keV argon energy for 15 s at each level and high-resolution XPS analysis was carried out. FIGS. 9A-9D show the XPS depth profiling of the Au, Zn, Ag, and O of the Au/ZnO/Ag. As expected, the binding energy of Zn, 0, and Ag are shifted with the increasing of etching time. The characteristic peak of Au is also shifted to higher binding energy, presumably due to the overlap of the Au 4f with the Zn3p. The increase in the etching time led to a decrease in the Au signal, while increasing the intensity of the Zn3p.

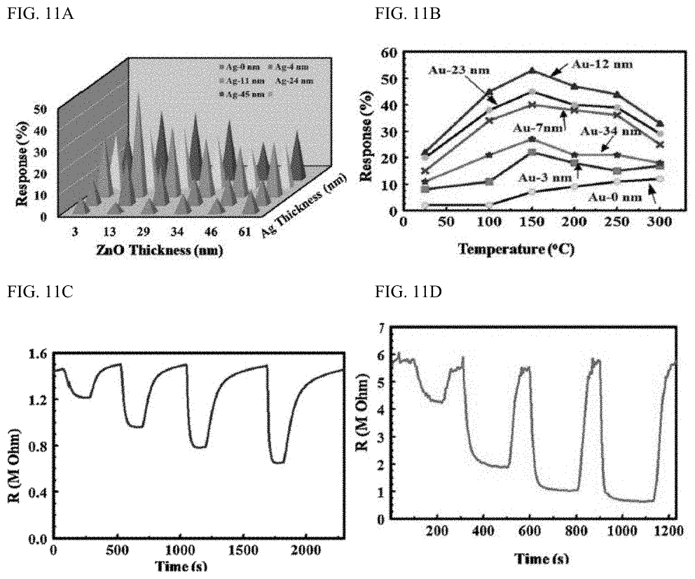

The optical properties of Ag film, Ag NPs, ZnO/Ag, and Au/ZnO/Ag film were characterized using UV-vis spectroscopy, and the obtained results are presented in FIGS. 10A-10B. As can be observed in FIG. 10A, no localized surface plasmon resonance (LSPR) for the Ag film was noticed. Whereas, an obvious characteristic peak of absorbance near 430 nm is observed in Ag NPs film, which is in good agreement with the characteristic LSPR of Ag NPs [D. K. Bhui, et. al., J. Mol. Liq., 2009, 145, 33-37; and B. Zargar, & A. Hatamie, Analyst, 2012, 137, 5334-5338, each of which is incorporated herein by reference in its entirety]. The UV-vis absorption spectrum of the Au decorated ZnO/Ag film showed three peaks at 372 nm, 420 nm, and 530 nm, corresponding to the characteristic ZnO, LSPR of Ag NPs, and LSPR of Au NPs, respectively [K. Xu, et. al., Opt. Mater. Express, 2019, 9; and K. Xu, Phys. Status Solidi, 2019, 216, 1800868, each of which is incorporated herein by reference in its entirety]. The peak at 420 nm is red-shifted LSPR of Ag NPs, which can be ascribed to the strong electromagnetic coupling between ZnO NPs coating Ag NPs film and the formation of ZnO-on-Ag NP film. The bandgap energy of the ZnO is usually estimated from the Tauc equation:

(ahv)ⁿ=A(hv−E_{g}) (2)

where α is the absorption coefficient calculated using the relation α=2.303/d, where d is the thickness of the film, A is a constant, h is the Planck's constant, and n is ½ for indirect bandgap and 2 for direct bandgap. FIG. 10B shows the bandgap of as-deposited ZnO and Au/ZnO/Ag films. As can be observed, the band gaps of ZnO and Au/ZnO/Ag films are calculated to be about 3.25 and 3.10 eV, respectively. The reduction of the bandgap of the Au/ZnO/Ag compared with that of as-deposited ZnO could be ascribed to the formation of structural defects i.e. oxygen vacancies, which might be produced during annealing the Au/ZnO/Ag sample at 600° C.

### Gas Sensing Properties

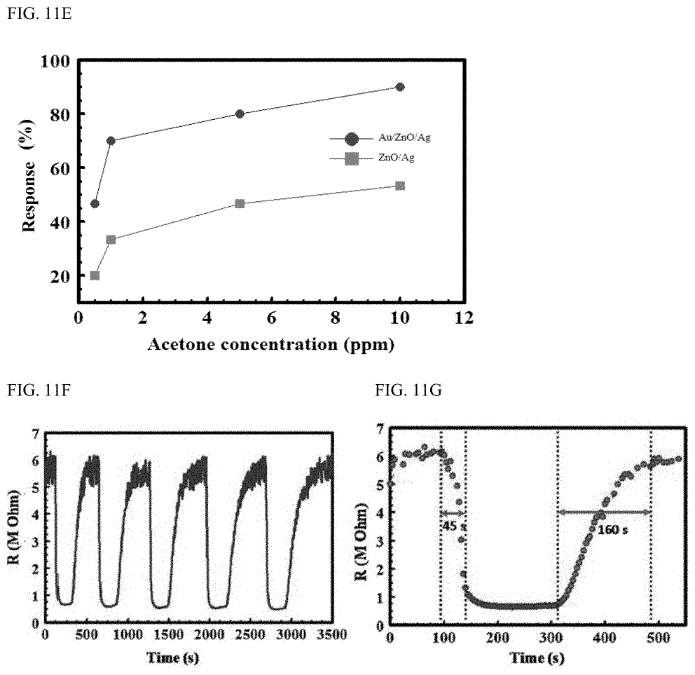

The sensing characteristic of chemiresistive gas sensors depends on several factors including preparation method and conditions, the materials which form the sensor, the working temperature, and the amount and the type of the noble metals. FIG. 11A shows the correlation between the response and thickness of ZnO as well as the density of Ag NPs. The thickness of Ag NPs and ZnO were varied from 0 to 45 nm (0 nm, 4 nm, 11 nm, 24 nm, 45 nm) and 3 to 61 nm (3 nm, 13 nm, 29 nm, 34 nm, 46 nm, 61 nm), respectively. For 1 ppm of acetone at 150° C., the optimum thickness of ZnO layers was discerned to be 13 nm, while Ag thickness was found to be 11 nm. The Au was deposited on the ZnO-on-Ag NP film and the optimum thickness of Au was also investigated by varying the Au thickness from 0 to 34 nm (0, 3, 7, 12, 23, and 34 nm). The experiments were conducted at different working temperatures ranging from room temperature (25° C.) to 300° C. The results are shown in FIG. 11B. The response increases with the increase in Au density up to 12 nm, and a further increase, such as 23 nm, led to a decrease in the performance. Moreover, a substantial decrease in the response was registered with 34 nm Au. The Au NPs with bigger size are likely to block the active sites of the ZnO film. The compositions with the optimal thickness (Ag—11 nm, ZnO—13 nm, and Au—12 nm) exhibiting the highest performance were selected as our standard assembly to further study the response of the Au/ZnO/Ag sensor towards acetone detection. Furthermore, the optimum working temperature that gives the highest response has been determined as 150° C. FIGS. 11C-11D show the dynamic response curves of ZnO/Ag and Au/ZnO/Ag in the range of 0.5˜10 ppm (0.5, 1, 5, and 10 ppm) at 150° C. As can be observed, the baseline of both sensors is stable and the sensors respond rapidly to acetone at 150° C. The sensing curves return to the initial value upon removal of acetone. Moreover, the gas response gradually increases with the increase in acetone concentration. The variation in response as a function of acetone concentrations in the presence of ZnO/Ag and Au/ZnO/Ag sensors is presented in FIG. 11E. The response of the Au/ZnO/Ag was almost two times higher with slower response and recovery time, as compared to the ZnO/Ag. For instance, at 150° C., the response of the ZnO/Ag toward 500 ppb acetone is ˜20%, while Au/ZnO/Ag response is ˜43%. The repeatability is another important quality of a gas sensor for any practical application. The repeatability of the Au/ZnO/Ag sensor was investigated by measuring 10 ppm of acetone at the working temperature of 150° C., and the obtained result is displayed in FIG. 11F. The result showed a consistent observation towards acetone sensing. The response/recovery curve is repeatable without any considerable changes. The sensor produces a similar response within 5 cycles, confirming its excellent repeatability. The response and recovery time are defined as the time required by the sensor for the variation in electrical resistance to reach 90% of the equilibrium value during the process of adsorption and desorption, respectively. The response and recovery time of the Au/ZnO/Ag sensors under 10 ppm of acetone at 150° C. were 45 and 160 s, respectively (FIG. 11G). The selectivity is another vital feature of gas sensors for practical applications. The selectivity of Au/ZnO/Ag was examined by exposing the sensor to a mixture of gases, including 5 ppm acetone, 100 ppm NO₂, 20,000 ppm 02, 1000 ppm H2, 200 ppm NH₃, 100 ppm CH₄, and 100 ppm C₄H₁₀. The measurement was conducted at 150° C., and the results are displayed in FIG. 11H. It is clear that the fabricated sensor exhibits excellent selectivity towards acetone with high response. Such findings strongly support the conclusion that the developed sensor holds great potential for the detection of acetone in practical applications. The long-term stability of the Au/ZnO/Ag is assessed by recording the response of the sensor repeatedly for 60 days. The response profiles of ZnO/Ag and Au/ZnO/Ag against time are compared in FIG. 11I. The response of both the materials remains almost intact for 60 days, indicating the long-term stability of the gas sensors. The acetone gas sensing properties (operating temperature, sensitivity, and response and recovery time) of the fabricated sensor of the present disclosure were compared with relevant literature data published in the last five years and displayed in Table 1. For example, the optimal operating temperature in the fabricated sensor of the present disclosure (150° C.) is much lower than many other sensors. In fact, only two sensing materials reported by Huang, et. al. and Cao, et. al. possess the same operating temperature [J. Huang, et. al., Sens. Actuators, B Chem., 2020, 310, 127129; and P. J. Cao, et. al., Appl. Surf. Sci., 2020, 518, 146223, each of which is incorporated herein by reference in its entirety]. In general, most acetone ZnO sensors reported in the literature (Table 1) operate at high temperatures (above 250° C.), which can shorten the lifetime and affect the long-term stability of the sensing device due to the changes in the properties of the nanostructured sensing material at elevated temperatures. Further, these high temperatures may be incompatible with certain gas mixtures or applications. Furthermore, among all these sensors in Table 1, only Huang, et. al. shows the ZnO acetone sensor in a thin film form. The most common technique used for the fabrication of ZnO thin films-based sensors is brush coating in which the as-fabricated sensing materials (nanorods, nanoparticles, nanosheets, etc.) are brushed on the surfaces of the substrates. Even though brush coating is an effective and simple method for fabrication of ZnO sensor with different morphologies and sizes, it has several shortcomings including, the limitation of the mass production that makes the fabricated sensors inappropriate for commercialization, the repeatability of the coating process is still challenging and the compatibility of the fabricated materials with substrates is relatively weak. The sputtering technique used in this work offers the advantages of producing high purity sensors, good reproducibility, and compatibility of the sensing materials with substrates, and easy scalability.

TABLE 1
Summary of acetone gas sensing properties of ZnO, and noble metal/ZnO sensors published in 2017-2022.

| | | | Optimum | Response | | |
|---|---|---|---|---|---|---|
| | | Acetone | Operating | toward | | |
| Sensing | | Detection | Temperature | detection | Response/Recovery | |
| Material | Morphology | Range (ppm) | (° C.) | limit | Time (s) | Ref |
| ZnO | Hierarchical | 5-35 | 300 | 45-90 | 42/10 @ 10 ppm | Guo 2017 |
| | urchin-like | | | | | |
| ZnO | Hierarchical | ~1-100 | 300 | 10-362 | 13/25 @ 50 ppm | Xie, et. al. 2017 |
| | architecture | | | | | |
| ZnO | Hollow | 10-100 | 300 | —/101 | 1/7 @100 ppm | Li, et. al. 2018 |
| | microspheres | | | | | |
| ZnO | Nanorods | 0.25-50 | 320 | 1.75-55 | 15/40 @ 50 ppm | Hadiyan, et. al. 2019 |
| ZnO | Nanofibers | 1-210 | 250 | ~1-180 | 75/25 @ 100 ppm | Du, et. al. 2020 |
| ZnO | Nanoplates | 50-125 | 450 | 11.76-125 | 23/637 @ 50 ppm | Van Duy, et. al. 2020 |
| ZnO | Elliptic | 10-300 | 280 | ~15-60 | 1/40 @ 10 ppm | Shi, et. al. 2020 |
| | microspheres | | | | | |
| Ag-doped | Nanoneedles | 10-200 | 370 | 1.3-3 | 10/21 @ 100 ppm | Al-Hadeethi, et. al. 2017 |
| ZnO | | | | | | |
| Au-doped | Thin film | 100-1000 | 280 | 60-130 | 22/39 @ 100 ppm | Deshwal, et. al. 2018 |
| ZnO | | | | | | |
| Pd-doped | Microflower | 5-100 | 370 | 5-81 | 10/5 @ 200 ppm | Zhang, et. al. 2019 |
| ZnO | | | | | | |
| Au/Pd- | Nanorods | 0.005-100 | 150 | ~1.5/100 (Au) | 8/5 (Au) | Huang, et. al. 2020 |
| doped ZnO | | | | ~1.7/75 (Pd) | 9/7 (Pd) | |
| Au-doped | Nanospheres | 0.5-200 | 170 | 2-350 | 27/18 @ 100 ppm | Cao, et. al. 2020 |
| ZnO | | | | | | |

### Table 1 References

[W. Guo, J. Mater. Sci. Mater. Electron., 2017, 28, 963-972; X. Xie, et. al., Ceram. Int., 2017, 43, 1121-1128; Y. Li, et. al., Sens. Actuators, B Chem., 2018, 273, 751-759; M. Hadiyan, et. al., J. Electroceramics., 2019, 42, 147-155; H. Du, et. al., ACS Appl. Mater. Interfaces, 2020, 12, 23084-23093; L. Van Duy, et. al., Mater. Today Commun., 2020, 25, 101445; W. Shi, et. al., Mater. Lett., 2020, 270, 127706; Y. Al-Hadeethi, et. al., Ceram. Int., 2017, 43, 6765-6770; M. Deshwal, & A. Arora, J. Mater. Sci. Mater. Electron., 2018, 29, 15315-15320; Y. H. Zhang, et. al., J. Phys. Chem. Solids, 2019, 124, 330-335; J. Huang, et. al., Sens. Actuators, B Chem., 2020, 310, 127129; and P. J. Cao, et. al., Appl. Surf. Sci., 2020, 518 146223, each of which is incorporated herein by reference in its entirety].

### Gas Sensing Mechanism

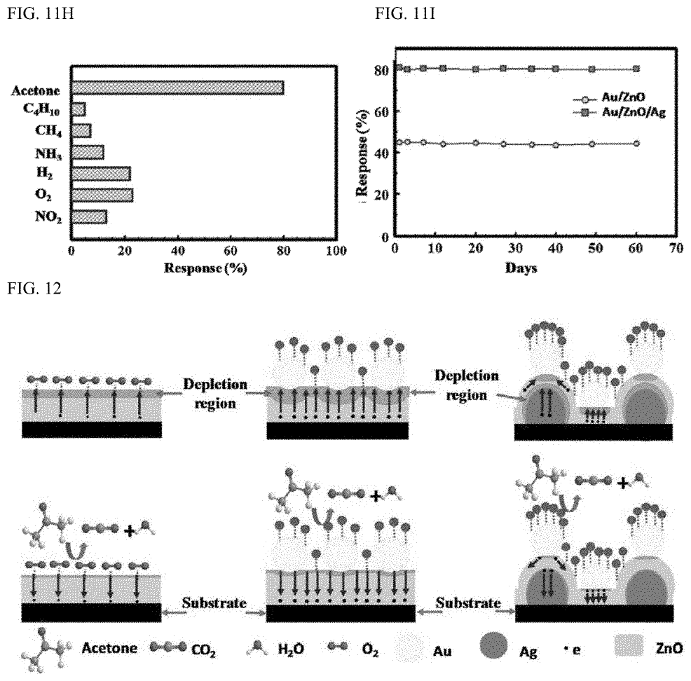

The basic principle of chemiresistive sensors is based on the electrical resistance change. When the surface of n-type metal oxides such as ZnO is surrounded by air, the atmospheric oxygen molecules tend to be adsorbed. This adsorption process is followed by the ionization of adsorbed oxygen molecules in the form of oxygen ions (O₂⁻, O⁻, O⁻²), and the type of ion formation is driven by the working temperature [Q. A. Drmosh, et. al., Sens. Actuators, B Chem., 2019, 290, 666-675, incorporated herein by reference in its entirety]. The electrons move from semiconductor metal oxides to O₂ molecules, creating a so-called “electron depletion region” at the interface. Such a depletion region is characterized by a low concentration of electrons in the n-type semiconductor, as depicted in FIG. 12. The deficiency of electrons at the metal oxide surfaces causes upward surface band bending, which in turn leads to an increase in the sensor resistance. When the n-type semiconductor sensing layer is exposed to the acetone (reducing gas), acetone is likely to be adsorbed on the surface and then react with the chemisorbed oxygen ions. The reaction between acetone and oxygen ions transfers the captured electrons back into the conduction band of the semiconductor. As a result, a reduction in the electron depletion region, the band bending, and the surface resistance can be expected. Decoration of thin films by Au NPs is shown herein to increase the electron density at the surface by increasing the depletion layer thickness to produce a significant enhancement in sensing properties of chemiresistive thin films. Two plausible models are usually adopted to explain the enhancement in the response of the noble metal/thin films sensor. One is the spillover mechanism and the other is based on the modulation of Fermi energy level (E_{f}) between the noble metal and the metal oxide [Q. A. Drmosh, & Z. H. Yamani, Ceram. Int., 2016, 42, 12378-12384; Q. A. Drmosh, & Z. H. Yamani, Appl. Surf. Sci., 2016, 375, 57-64; Q. A. Drmosh, et. al., Vacuum, 2018, 156, 68-77; and M. R. Alenezi, et. al., RSC Adv., 2015, 5, 103195-103202, each of which is incorporated herein by reference in its entirety]. In Au/ZnO/Ag, the formation of two depletion regions is expected at the two sides of the ZnO film due to the difference in work-function of Ag and ZnO and Au and ZnO. The energy band bending between Ag and ZnO, and Au and ZnO induce electron drift from Ag and ZnO to Au NPs, as confirmed by the XPS. This electron drifting continues until the Ef of the three compounds reaches equilibrium. When the equilibrium is maintained, the formation of a thick depletion layer in Ag and ZnO with more electron density near Au NPs is likely. Such surface rearrangement favors the adsorption of 02 molecules. The highest adsorption can be obtained by achieving optimal synergy amongst ZnO, Au, and Ag, which is dependent on the thickness of ZnO and the amount of Au and Ag. When acetone is introduced, the adsorbed oxygen ions react with acetone molecules transforming the thicker region into a thinner electron depletion layer. The sensor response towards acetone can be represented by the chemical equation as follows:

CH₃COCH₃(gas)+O₂⁻→H₂O(gas)+CO₂(gas)+2e⁻

The thickness (width) of the depletion layer (W) is represented by the following equation:

$$W = {{L}_{d} ( \frac{q {V}_{s}}{k T} )}^{1 / 2} \qquad ( 4 )$$

where L_{d}, K, q, Vₛ and T are the Debye length, Boltzmann constant, elementary charge, band bending, and operating temperature, respectively. The thickness of the depletion layer can be influenced by different factors, such as the preparation method, polymorphic forms, doping, decoration, and the shell layer [J. Liu, et. al., Appl. Phys. A Mater. Sci. Process., 2016, 122 1-7; C. C. Li, et. al., Appl. Phys. Lett., 2007, 91, 032101, each of which is incorporated herein by reference in its entirety]. A sensing layer thinner than L_{d} results in a layer that is fully electrically depleted. On the contrary, the charge carriers will be mostly restricted in the sensing layer if the thickness of the sensing layer is thicker or equal to the L_{d}. Based on the reported carrier concentration of ZnO, the Debye length of ZnO is calculated to be 15 nm [E. Wongrat, et. al., Sens. Actuators, B Chem., 2012, 171-172, 230-237, incorporated herein by reference in its entirety]. Hence, the thickness of the ZnO layer (3 and 13 nm) is less than the Debye length of ZnO, resulting in the full electron depletion of ZnO. However, ZnO having 3 nm thickness showed a very high electrical resistance presumably due to the formation of ZnO islands rather than a continuous layer. As a result, the ZnO with 3 nm layer thickness exhibited a significantly lower response as compared to the ZnO with 13 nm layer thickness, which showed the maximum response (see FIG. 11A). As the thickness of the ZnO films increased to 34, 46, and 61 nm, which exceeded the Debye length, the bulky part of the ZnO films failed to influence the surface reaction, resulting in a lower response.

The remarkable enhancement in the acetone sensing performance of the Au/ZnO/Ag sensors is interpreted as follows: First, Au is a chemical sensitizer capable of providing more adsorbed oxygen molecules onto ZnO/Ag core-shell through the spillover effect. The larger amount of adsorbed oxygen species can serve as more reaction sites for acetone available on the Au/ZnO/Ag surface, thereby exhibiting a higher response. Second, double activation of the optimum thickness of ZnO layer resulted in thicker depletion region in air and thinner in acetone environment which contributes significantly to the improved acetone sensing properties of Au/ZnO/Ag sensor. Furthermore, the higher surface area of the Au/ZnO/Ag film compared with that of ZnO film provides abundant active sites, which in turn lead to promote the gas diffusion and absorption on the ZnO surface.

1: A gas sensor, comprising:
a substrate; and
an active material, comprising:
first noble metal nanoparticles disposed on the substrate;
a zinc oxide layer disposed on the first noble metal nanoparticles and the substrate; and
second noble metal nanoparticles disposed on the zinc oxide layer;
wherein the zinc oxide layer prevents contact between the second noble metal nanoparticles and the first noble metal nanoparticles and between the second noble metal nanoparticles and the substrate.

2: The gas sensor of claim 1, wherein the first noble metal nanoparticles have a mean particle size of 50 to 300 nm.

3: The gas sensor of claim 1, wherein the first noble metal nanoparticles are silver nanoparticles.

4: The gas sensor of claim 1, wherein the zinc oxide layer has a thickness of 1 to 75 nm.

5: The gas sensor of claim 1, wherein the zinc oxide layer comprises wurtzite zinc oxide which is crystalline by PXRD.

6: The gas sensor of claim 1, wherein the second noble metal nanoparticles have a mean particle size of 25 to 250 nm.

7: The gas sensor of claim 1, wherein the second noble metal nanoparticles are gold nanoparticles.

8: The gas sensor of claim 1, wherein the active material has a band gap of 2.80 to 3.20 eV.

9: m 9: A method of forming the gas sensor of claim 1, the method comprising:
sputtering a first film of a first noble metal onto the substrate to form a first film-comprising material;
annealing the first film-comprising material to form the first noble metal nanoparticles;
depositing the zinc oxide layer on the first noble metal nanoparticles by sputtering;
sputtering a second film of a second noble metal onto the zinc oxide layer to form a second film-comprising material; and
annealing the second film-comprising material to form the gas sensor.

10: 10: The method of claim 9, wherein the first noble metal is silver.

11: The method of claim 9, wherein the second noble metal is gold.

12: The method of claim 9, wherein:
the first film has a thickness of 1 to 75 nm; and
the second film has a thickness of 1 to 50 nm.

13: The method of claim 9, wherein the first film-comprising material is annealed at 450 to 650° C.: under inert atmosphere.

14: The method of claim 9, wherein the second film-comprising material is annealed at 500 to 700° C.: under inert atmosphere.

15: A method of detecting the presence of a ketone in a gas sample, the method comprising:
applying a voltage to the gas sensor of claim 1;
exposing the gas sample to the gas sensor; and
detecting a change in the electrical properties of the gas sensor to determine whether a ketone is present or absent in the gas sample.

16: The method of claim 15, wherein the ketone has a molecular weight of less than 300 g/mol.

17: The method of claim 16, wherein the ketone is acetone.

18: The method of claim 15, wherein the exposing is performed at 25 to 300° C.

19: The method of claim 15, wherein the method has a lower detection limit of 0.1 to 1 ppm of ketone.

20: The method of claim 17, wherein the method has a lower detection limit of 0.1 to 1 ppm of acetone.
